(19) United States
(12) Patent Application Publication
(10) Pub. No.: US 2026/0315727 A1
(43) Pub. Date: Oct. 15, 2026

(54) DEVICES, SYSTEMS, AND METHODS FOR POSITIVE AIRWAY PRESSURE THERAPY

(71) Applicant: Rabih Nassif, North Tustin, CA (US)

(72) Inventor: Rabih Nassif, North Tustin, CA (US)

(21) Appl. No.: 19/464,464

(22) Filed: Jan. 29, 2026

Related U.S. Application Data
(60) Provisional application No. 63/752,588, filed on Jan. 31, 2025.

(51) Int. Cl. A61M 16/20

(52) U.S. Cl. CPC A61M 16/208

(57) ABSTRACT

Devices and methods for controlling airflow through a positive airway pressure (PAP) system are disclosed herein. According to some embodiments, the present technology includes an airflow controller comprising a conduit defining a lumen with a valve within the lumen and a venting device defining a cavity and an outlet with a diaphragm within the cavity. The valve is configured to permit pressurized air to flow from a pressurized air source to a patient during inspiration and prevent exhaled air from flowing beyond the valve from the patient towards the pressurized air source during expiration at a pressure about equal to that of the pressurized air. The diaphragm is configured to allow the exhaled air to flow out of the outlet during expiration and prevent the pressurized air from leaking through the outlet during inspiration.

### CROSS-REFERENCE TO RELATED APPLICATIONS

This application claims the benefit of priority to U.S. Provisional Application No. 63/752,588, filed Jan. 31, 2025, which is incorporated herein by reference in its entirety.

### TECHNICAL FIELD

The present technology relates to devices, systems, and methods for positive airway pressure therapy. Various embodiments of the present technology are directed to devices and methods for controlling airflow through a positive airway pressure system.

### BACKGROUND

Positive airway pressure (PAP) therapy delivers pressurized air to the lungs of a patient suffering from respiratory dysfunction to assist or replace the patient's spontaneous respiration. PAP therapy is an effective treatment for a variety of acute and chronic conditions, including obstructive sleep apnea (OSA). During sleep, the airway of a patient with OSA is obstructed and/or collapses so that airflow into the patient's lungs is partially or completely blocked and the patient's blood oxygenation decreases. Insufficient oxygenation can severely damage the patient's organs, particularly the brain and heart, and is a serious medical condition. A patient's body detects these reductions in blood oxygenation and initiates a series of physiological responses intended to bring more oxygen into the body and move oxygen throughout the body more quickly. Such physiological responses include increased blood pressure and heart rate, greater dilation of the heart, and sudden activation of the muscles controlling patency of the airway. While these reflexive physiological responses may increase the patient's blood oxygenation, they also stress the patient's cardiovascular system, frequently arouse the patient from sleep, and negatively affect the patient's health and quality of life.

A patient suffering from OSA may use a PAP system during sleep to prevent obstruction and/or collapse of the airway and to facilitate airflow into the lungs. With reference to FIG. 1, a conventional PAP system 100 includes a pressurized air source 102, a patient interface 104 configured to be coupled to the patient's nose and/or mouth, and a conduit 106 extending from a first end portion at the pressurized air source 102 to a second end portion at the patient interface 104. The pressurized air source 102 is configured to compress air from the environment 101 into pressurized air 103 at a pressure greater than the pressure within the alveoli of the lungs during inspiration. The resulting pressure gradient causes the pressurized air 103 to travel in a first direction along a primary airflow path defined by the conduit 106 and patient interface 104 from the pressurized air source 102 to the patient's nose and/or mouth and further into the patient's airway and lungs, thus facilitating the transport of oxygen into the patient's body. The pressurized air 103 also pneumatically splints the airway open to prevent collapse and/or obstruction of the airway. The pressurized air source 102 can be configured to generate pressurized air 103 at a single pressure throughout the patient's entire respiratory cycle (e.g., known as continuous positive airway pressure (CPAP)). Bi-level positive airway pressure (BiPAP) systems generate pressurized air 103 at a higher pressure during inspiration and a lower pressure during expiration to facilitate movement of air out of the lungs during expiration. Automatic positive airway pressure (APAP) systems adjust the pressure of the pressurized air 103 in response to sensed changes in the patient's respiration. Regardless of the type of PAP system 100, the pressurized air source 102 still generates pressurized air 103 during expiration; however, the pressure within the alveoli exceeds the pressure of the pressurized air 103, and the patient expels carbon dioxide-rich exhaled air 105 from their lungs, airway, and nose and/or mouth into the patient interface 104 and thereby into the primary airflow path. The exhaled air 105 travels in a second direction along the primary airflow path until the exhaled air 105 vents from the PAP system 100 to the environment via outlets in the patient interface 104 and/or the conduit 106.

Venting the exhaled air 105 to the environment is a significant challenge that is poorly addressed by conventional PAP systems 100. The outlets are typically located along the primary airflow path so that pressurized air 103 also vents through the outlets, which undesirably alters the pressure of the pressurized air that reaches the patient and diminishes the efficacy and comfort of the PAP therapy. Consequently, the size and number of outlets in conventional PAP systems are limited to attempt to limit leakage of pressurized air 103. Limiting the size and number of outlets, however, also limits the volume of exhaled air that can be vented through the outlets at a given time. The efficiency at which exhaled air 105 vents through the outlets is also dependent on the pressure of the pressurized air 103. Exhaled air 105 vents through the outlets more efficiently when the pressurized air 103 is compressed to higher pressures; however, high pressures are also associated with negative side effects for the patient (e.g., discomfort, aerophagia, etc.) and some patients only require low pressures to receive therapeutic benefits of the PAP therapy.

Exhaled air 105 that is not vented from the PAP system 100 mixes with the pressurized air 103 and is rebreathed by the patient, leading to hypercapnia, an excess buildup of carbon dioxide within the patient. Chemosensitive neurons detect rising carbon dioxide levels and cause the patient to hyperventilate (e.g., breathing rapidly and deeply) in an effort to purge the excess carbon dioxide from the body and prevent the serious adverse effects of hypercapnia. However, during hyperventilation large volumes of air are exhaled in short, frequent breaths. As previously noted, the volume of air that can be vented at a given time is limited by the size and number of the outlets and thus, the short, frequent breaths are poorly vented by the PAP system 100. A positive feedback cycle thus begins in which carbon dioxide from the exhaled air 105 accumulates in the PAP system 100 and is rebreathed by the patient, the patient hyperventilates, more carbon dioxide accumulates in the PAP system 100 and is rebreathed, and the intensity of hyperventilation increases until the patient is vigorously aroused from sleep in a state of respiratory distress. In this manner, conventional PAP systems 100 can exacerbate the deoxygenation, cardiovascular stress, poor sleep, and reduction in quality of life caused by OSA.

The exhaled air 105 expelled into the PAP system 100 is also laden with water and microbes. When the exhaled air 105 travels through and/or collects within the patient interface 104 and/or conduit 106, as occurs with conventional PAP systems, the water and microbes are deposited within these components. The microbes can proliferate within the moist environment of the components and contaminate the pressurized air 103 that travels through these same components to the patient. Consequently, the patient interface 104 and/or conduit 106 of conventional PAP systems 100 must be cleaned and/or replaced frequently.

Thus, a need exists for devices, systems, and methods to efficiently vent exhaled air from PAP systems without interfering with the pressure(s) required for PAP therapy.

### SUMMARY

The subject technology is illustrated, for example, according to various aspects described below, including with reference to FIGS. 1-4B. Various examples of aspects of the subject technology are described as numbered clauses (1, 2, 3, etc.) for convenience. These are provided as examples and do not limit the subject technology.

1. A device for venting air exhaled by a patient into a lumen of a conduit of a positive airway pressure system, the device comprising:
a wall defining a cavity comprising a first chamber and a second chamber, wherein the wall defines a first inlet fluidically coupled to the first chamber, a second inlet fluidically coupled to the second chamber, and an outlet fluidically coupled to the second chamber, wherein the first and second inlets are configured to be fluidically coupled to the lumen; and
a diaphragm positioned within the cavity and defining an interior region fluidically coupled to the first chamber,
wherein, when a first pressure within the interior region is greater than a second pressure within the second chamber during inspiration, the diaphragm extends across the outlet and prevents airflow through the outlet, and
wherein, when the first pressure is less than the second pressure during expiration, the diaphragm no more than partially extends across the outlet and permits the exhaled air to flow through the outlet from the second chamber.

2. The device of Clause 1, wherein the interior region and the first chamber are configured to receive pressurized air from a pressurized air source of the positive airway pressure system.

3. The device of any one of the preceding Clauses, wherein the second chamber is configured to receive the exhaled air during expiration.

4. The device of any one of the preceding Clauses, wherein the diaphragm is configured to contact an inner surface of the wall proximate the outlet during inspiration.

5. The device of any one of the preceding Clauses, wherein the diaphragm is configured to be spaced apart from an inner surface of the wall proximate the outlet during expiration.

6. The device of any one of the preceding Clauses, wherein a volume of the interior region is greater during inspiration than expiration.

7. The device of any one of the preceding Clauses, wherein the diaphragm is configured to transform between a first configuration during inspiration and a second configuration during expiration.

8. The device of any one of the preceding Clauses, wherein the transforming comprises deforming, rotating, and/or translating.

9. The device of any one of the preceding Clauses, wherein the wall is substantially tubular.

10. The device of any one of the preceding Clauses, wherein the wall has a closed first end and a closed second end opposite the first end along a length of the wall.

11. The device of any one of the preceding Clauses, wherein the diaphragm is secured to the wall by a retainer within the cavity.

12. The device of any one of the preceding Clauses, wherein the retainer defines an aperture fluidically coupling the interior region to the first chamber.

13. The device of any one of the preceding Clauses, wherein the diaphragm comprises a closed end and an open end.

14. The device of any one of the preceding Clauses, wherein the retainer is positioned within the open end of the diaphragm.

15. The device of any one of the preceding Clauses, wherein the retainer comprises a disc.

16. A device for controlling airflow between a pressurized air source of a positive airway pressure system and a patient, the device comprising:
a conduit defining a lumen extending along a length of the conduit, the conduit having a first end portion configured to be fluidically coupled to the pressurized air source and a second end portion configured to be fluidically coupled to the patient; and
a valve positioned within the lumen and configured to transition between:
   an open configuration in which the valve allows pressurized air from the pressurized air source to flow through the lumen from the first end portion of the conduit to the second end portion of the conduit during inhalation; and
   a closed configuration in which the valve prevents exhaled air from the patient from flowing through the lumen from the second end portion of the conduit to the first end portion of the conduit during exhalation.

17. The device of any one of the preceding Clauses, wherein the valve does not substantially extend across the lumen in the open configuration and does substantially extend across the lumen in the closed configuration.

18. The device of any one of the preceding Clauses, wherein the valve is angled relative a longitudinal axis of the conduit by a first angle in the open configuration and a second angle in the closed configuration, and wherein the second angle is greater than the first angle.

19. The device of any one of the preceding Clauses, wherein the valve has a first end portion secured to the conduit and a free second end portion.

20. The device of any one of the preceding Clauses, wherein the valve is configured to rotate about the first end portion when transitioning between the open and closed configurations.

21. The device of any one of the preceding Clauses, wherein the first end portion is configured to be directly coupled to the pressurized air source.

22. The device of any one of the preceding Clauses, wherein the second end portion is configured to be directly coupled to the patient interface.

23. The device of any one of the preceding Clauses, wherein the first end portion is configured to be coupled to a second conduit extending between the first end portion and the pressurized air source.

24. The device of any one of the preceding Clauses, wherein the second end portion is configured to be coupled to a third conduit extending between the first end portion and the patient interface.

25. The device of any one of the preceding Clauses, wherein the second end portion is configured to be coupled to a third conduit extending between the first end portion and the patient.

26. The device of any one of the preceding Clauses, wherein the conduit comprises a first shaft extending from a first end portion to a second end portion and defining a first shaft lumen and a second shaft extending from a first end portion to a second end portion and defining a second shaft lumen, and wherein the first shaft is configured to be positioned at least partially within the second shaft lumen.

27. The device of any one of the preceding Clauses, wherein the first shaft is configured to be positioned within the second shaft lumen such that the second end portion of the first shaft is positioned between the first end portion of the second shaft and the second end portion of the second shaft.

28. The device of any one of the preceding Clauses, wherein an end surface of the first shaft at the second end portion of the first shaft is angled relative to a longitudinal axis of the first shaft.

29. The device of any one of the preceding Clauses, wherein the second end portion of the first shaft provides a seat for the valve when the valve is in the closed configuration.

30. A device for controlling airflow between a pressurized air source of a positive airway pressure system and a patient, the device comprising:
a conduit defining a lumen extending along a length of the conduit, the conduit having a first end portion configured to be fluidically coupled to the pressurized air source and a second end portion configured to be fluidically coupled to the patient;
a valve positioned within the lumen, wherein the valve is configured to (i) permit pressurized air to flow through the lumen from the pressurized air source to the patient during inspiration, and (ii) prevent exhaled air from flowing through the lumen past the valve from the patient towards the pressurized air source during expiration; and
a venting device comprising:
   a wall defining a cavity comprising a first chamber and a second chamber, each of the first and second chambers being configured to be fluidically coupled to the lumen, wherein the wall defines an outlet fluidically coupling the second chamber to an environment external of the wall; and
   a diaphragm positioned within the cavity and defining an interior region fluidically coupled to the first chamber, wherein the diaphragm is configured to (i) prevent the pressurized air from flowing from the second chamber to the environment through the outlet during inspiration, and (ii) permit the exhaled air to flow from the second chamber to the environment through the outlet during expiration.

31. The device of any one of the preceding Clauses, wherein the diaphragm is configured to transform between a first configuration assumed during inspiration and a second configuration assumed during expiration.

32. The device of any one of the preceding Clauses, wherein a volume of the interior region defined by the diaphragm is greater when the diaphragm is in the first configuration than when the diaphragm is in the second configuration.

33. The device of any one of the preceding Clauses, wherein, when the diaphragm is in the first configuration during inspiration, the diaphragm is positioned over the outlet and in contact with an inner surface of the wall.

34. The device of any one of the preceding Clauses, wherein, when the diaphragm is in the second configuration during expiration, the diaphragm is spaced apart from the inner surface of the wall.

35. The device of any one of the preceding Clauses, wherein, when the diaphragm is in the second configuration during expiration, the diaphragm is not positioned over the outlet.

36. The device of any one of the preceding Clauses, wherein the diaphragm collapses when transforming from the first configuration to the second configuration.

37. The device of any one of the preceding Clauses, wherein the valve is configured to transition between an open configuration assumed during inspiration and a closed configuration during expiration.

38. The device of any one of the preceding Clauses, wherein, when the valve is in the open configuration during inspiration, the valve does not substantially extend across the lumen of the conduit.

39. The device of any one of the preceding Clauses, wherein, when the valve is in the closed configuration during expiration, the valve substantially extends across the lumen of the conduit.

40. A device for controlling airflow between a pressurized air source of a positive airway pressure system and a patient, the device comprising:
a conduit having an open first end configured to be fluidically coupled to the pressurized air source and an open second end configured to be fluidically coupled to the patient, the conduit defining a lumen comprising a first region proximate the first end and a second region proximate the second end;
a valve positioned within the lumen between the first and second regions of the lumen;
a wall having a closed first end and a closed second end, the wall defining a cavity comprising a first chamber fluidically coupled to the first region of the lumen via a first inlet and a second chamber fluidically coupled to the second region of the lumen via a second inlet, the second chamber being fluidically coupled to an environment external of the wall via an outlet defined by the wall; and
a diaphragm positioned within the cavity between the first and second chambers,
wherein, during inspiration, a pressure at the first end of the conduit is greater than a pressure at the second end of the conduit, the valve permits pressurized air to flow from the first region of the lumen to the second region of the lumen, and the diaphragm prevents the pressurized air from flowing from the second chamber to the environment via the outlet, and
wherein, during expiration, the pressure at the second end of the conduit is greater than the pressure at the first end of the conduit, the valve prevents exhaled air from flowing from the second region of the lumen to the first region of the lumen, and the diaphragm allows the exhaled air to flow from the second chamber to the environment via the outlet.

41. The device of any one of the preceding Clauses, wherein, during inspiration, a pressure within the first region of the lumen and a pressure within the second region of the lumen correspond to the pressure at the first end of the conduit.

42. The device of any one of the preceding Clauses, wherein, during expiration, a pressure within the first region of the lumen corresponds to the pressure at the first end of the conduit and a pressure within the second region of the lumen corresponds to the pressure at the second end of the conduit.

43. The device of any one of the preceding Clauses, wherein a pressure within the first chamber corresponds to the pressure at the first end of the conduit during inspiration and expiration.

44. The device of any one of the preceding Clauses, wherein a pressure within the second chamber corresponds to the pressure at the second end of the conduit.

45. The device of any one of the preceding Clauses, wherein during inspiration, the valve extends across the lumen of the conduit, and wherein during expiration, the valve does not extend across the lumen of the conduit.

46. The device of any one of the preceding Clauses, wherein during inspiration, the diaphragm extends across the outlet, and wherein during expiration, the diaphragm does not extend across the outlet.

47. A device for venting air exhaled by a patient into a lumen of a conduit of a positive airway pressure system, the device comprising:
a wall defining a cavity comprising a first chamber and a second chamber, wherein the wall defines a first inlet in fluid communication with the first chamber, a second inlet in fluid communication with the second chamber, and an outlet in fluid communication with the second chamber, wherein the first and second inlets are configured to be fluidically coupled to a lumen of a conduit of a positive airway pressure system; and
a diaphragm positioned within the cavity and defining an interior region in fluid communication with the first chamber,
wherein, when a first pressure within the interior region of the diaphragm is greater than a second pressure within the second chamber during inspiration, the diaphragm extends across the outlet and prevents airflow through the outlet, and
wherein, when the first pressure is less than the second pressure during expiration, the diaphragm no more than partially extends across the outlet and permits the exhaled air to flow through the outlet from the second chamber, and allows the first and second pressures to equalize.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 schematically illustrates airflow through a conventional PAP system.

FIG. 2A illustrates a PAP system in accordance with the present technology.

FIG. 2B schematically illustrates airflow through the PAP system of FIG. 2A.

FIGS. 3A and 3B are cross-sectional views of an airflow controller in accordance with the present technology. FIGS. 3A and 3B illustrate airflow through the airflow controller during an inspiratory phase of a patient's respiratory cycle and an expiratory phase of a patient's respiratory cycle, respectively.

FIGS. 4A and 4B are cross-sectional views of an airflow controller in accordance with the present technology. FIGS. 4A and 4B illustrate airflow through the airflow controller during an inspiratory phase of a patient's respiratory cycle and an expiratory phase of a patient's respiratory cycle, respectively.

### DETAILED DESCRIPTION

The present technology relates to devices and methods for controlling airflow through a PAP system. Various embodiments of the present technology are directed to an airflow controller configured to control the flow of exhaled air through a PAP system to vent the exhaled air to the environment, prevent or limit mixing of the exhaled air with pressurized air, and/or prevent or limit the exhaled air from contaminating one or more components of the PAP system. According to various embodiments, the airflow controller can comprise a conduit with a valve positioned within the lumen of the conduit. The conduit lumen defines at least a portion of a primary airflow path between a pressurized air source and a patient and the valve is configured to permit airflow in a single direction along the primary airflow path. For example, the valve can be configured to permit the flow of pressurized air from the pressurized air source to the patient while preventing the flow of exhaled air from the patient towards the pressurized air source beyond the valve. The valve can therefore be configured to prevent exhaled air from traveling along a significant length of the primary airflow path, which can prevent or limit mixing of the exhaled air with the pressurized air and contamination of the PAP system by the exhaled air. In some embodiments, the airflow controller can include a venting device defining a vent airflow path and at least one outlet for venting the exhaled air from the PAP system. The venting device can be configured to be fluidically coupled to the conduit so that exhaled air travels along the vent airflow path separate from the pressurized air within the primary airflow path. According to various embodiments, the venting device includes a pressure-responsive diaphragm configured to extend over and block the outlet during inspiration and configured to move away from the outlet and allow exhaled air to pass through the outlet during expiration. Consequently, the venting device can include more and/or larger outlets to vent the exhaled air more efficiently without excessive leakage of the pressurized air.

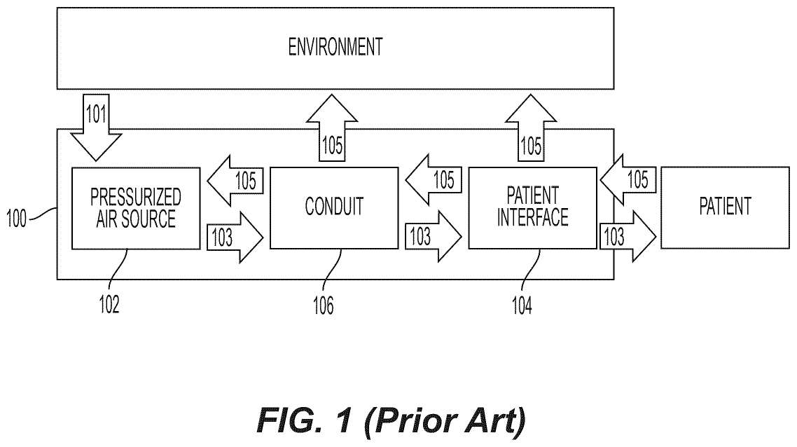

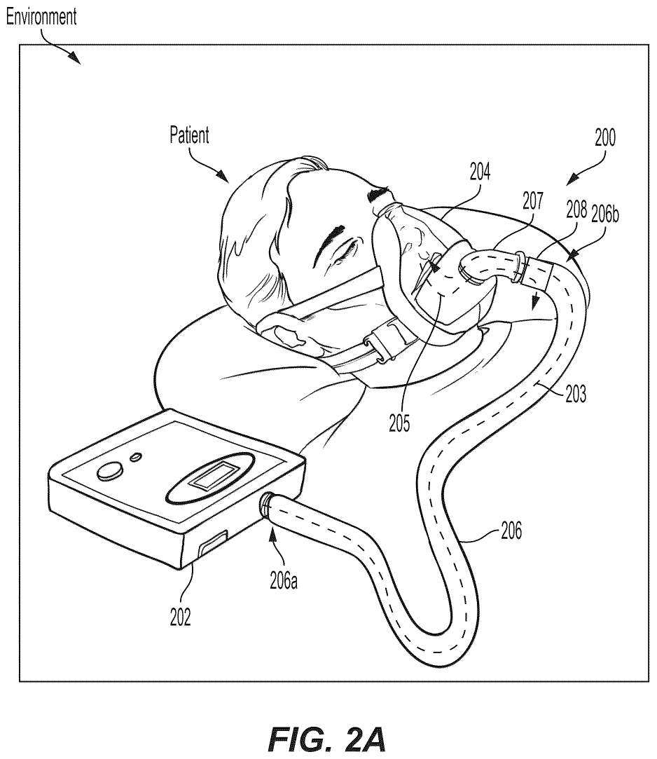

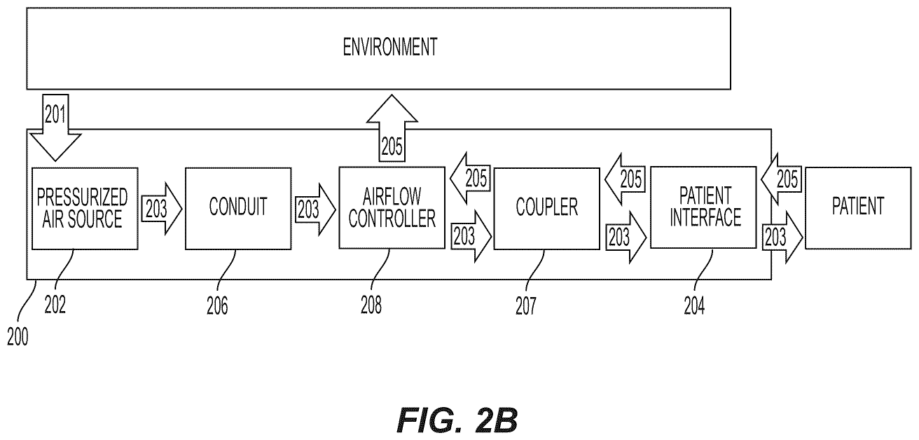

FIG. 2A illustrates a PAP system 200 (also referred to as the “system 200” herein) and FIG. 2B schematically illustrates airflow through the system 200 in accordance with various embodiments of the present technology. The system 200 can be similar to the PAP system 100 described with reference to FIG. 1, except as detailed below. For example, similar to the PAP system 100, the system 200 can include a pressurized air source 202 configured to compress environmental air 201 into pressurized air 203, a patient interface 204, and a conduit 206 extending from a first end portion 206a configured to be fluidically coupled to the pressurized air source 202 to a second end portion 206b configured to be fluidically coupled to the patient interface 204. In some embodiments, for example as shown in FIG. 2A, the system 200 can include a coupler 207 positioned between and fluidically coupling the conduit 206 to the patient interface 204. Unlike conventional PAP systems, the system 200 includes an airflow controller 208 positioned between the pressurized air source 202 and the patient and configured to control the flow of pressurized air 203 and/or exhaled air 205 between the pressurized air source 202 and patient.

In some embodiments, for example as shown in FIGS. 2A and 2B, the airflow controller 208 is positioned at or proximate to the second end portion 206b of the conduit 206 so that the airflow controller 208 is positioned between the conduit 206 and coupler 207 and/or the patient interface 204. Alternatively, the airflow controller 208 can be positioned between along the length of the conduit 206 between the first end portion 206a and the second end portion 206b of the conduit 206. In some embodiments, the coupler 207 can include or be replaced by the airflow controller 208 with the airflow controller 208 positioned between and coupled to the conduit 206 and the patient interface 204. In some embodiments, the patient interface 204 can include the airflow controller 208. For example, the airflow controller 208 can be integrated into a mask configured to be worn by the patient during use of the system 200. It may be advantageous to position the airflow controller 208 at or proximate to the patient interface 204 (e.g., at the patient interface 204, at the coupler 207, at the second end portion 206b of the conduit 206, etc.) to limit the length of the conduit 206 along which the exhaled air 205 travels before reaching the airflow controller 208, which can limit mixing of the exhaled air 205 with the pressurized air 203 and/or contamination of the conduit 206. Still, the airflow controller 208 can be positioned at any location along the primary airflow path between the pressurized air source 202 and the patient.

Collectively, the conduit 206, the airflow controller 208, the coupler 207 (if included), and the patient interface 204 define a primary airflow path along which pressurized air 203 can flow in a first direction from the pressurized air source 202 to the patient. The primary airflow path can be configured to receive exhaled air 205 expelled by the patient into the patient interface 204 and the exhaled air 205 can travel along a portion of the primary airflow path in a second direction different than (e.g., opposite to, angled relative to, etc.) the first direction and towards the pressurized air source 202. However, unlike conventional PAP systems, the airflow controller 208 can prevent the exhaled air 205 from flowing along the primary airflow path in the second direction beyond the airflow controller 208. To this end, the airflow controller 208 can define a vent airflow path along which the exhaled air 205 flows out of the PAP system 200 and into the environment. Thus, the airflow controller 208 can be configured to prevent or limit passage of exhaled air 205 along the primary airflow path, which in turn prevents or limits mixing of the exhaled air 205 with the pressurized air 203 and contamination of the components of the system 200 with water and microbes in the exhaled air 205.

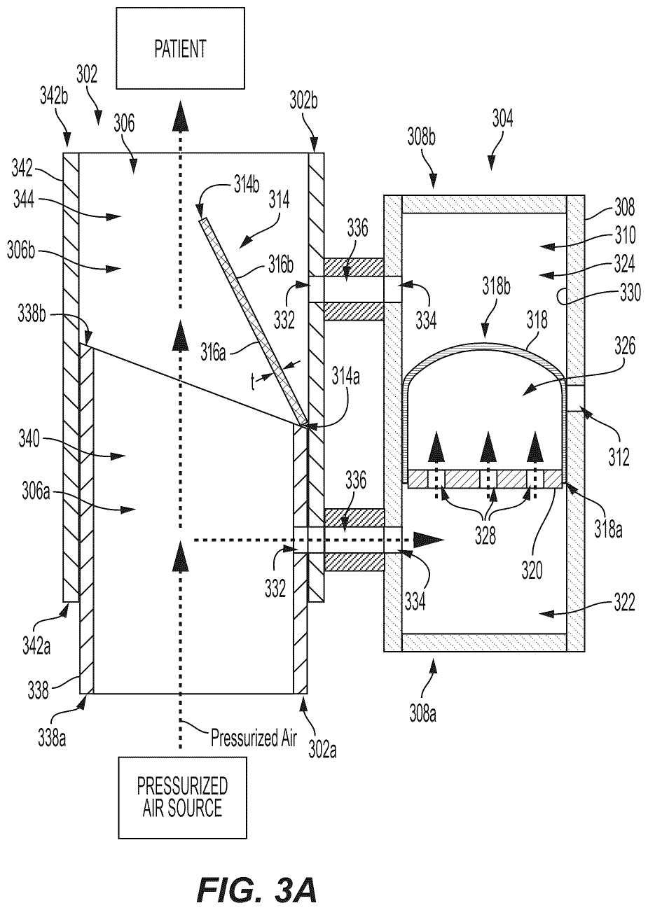

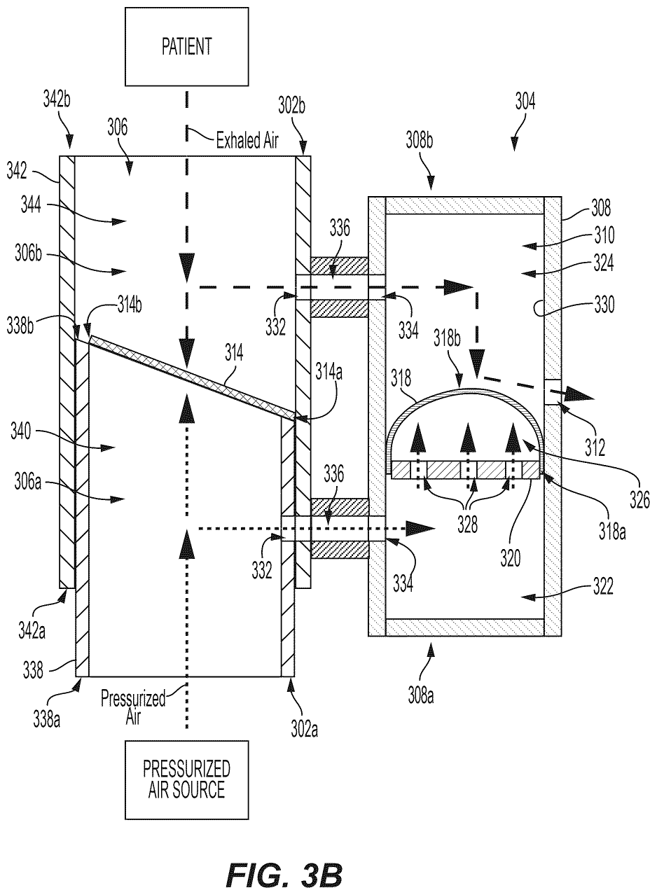

FIGS. 3A and 3B are side cross-sectional views of an airflow controller 300 in accordance with various embodiments of the present technology. The airflow controller 300 of FIGS. 3A and 3B can be similar to the airflow controller 208 described with reference to FIGS. 2A and 2B and/or the airflow controller 208 can be similar to the airflow controller 300. Additionally or alternatively, the PAP system 200 of FIGS. 2A and 2B can incorporate the airflow controller 300 of FIGS. 3A and 3B and/or the airflow controller 300 of FIGS. 3A and 3B can be used with any suitable PAP system. FIG. 3A illustrates the airflow controller 300 during inspiration and FIG. 3B illustrates the airflow controller 300 during expiration.

Referring to FIGS. 3A and 3B, the airflow controller 300 can comprise a conduit 302 and a venting device 304. The conduit 302 extends from a first end portion 302a to a second end portion 302b and defines a lumen 306 defining an airflow path through the conduit 302. The airflow path defined by the lumen 306 can comprise at least a portion of a primary airflow path for delivering pressurized air from a pressurized air source of a PAP system to a patient. The venting device 304 can include a wall 308 with a closed first end portion 308a and a closed second end portion 308b defining a cavity 310 and an outlet 312 fluidically coupling the cavity 310 to the environment. The cavity 310 can be configured to be fluidically coupled to the lumen 306 to define a vent airflow path for venting exhaled air from the lumen 306 to the environment.

According to various embodiments, the airflow controller 300 can include a valve 314 configured to be positioned within the lumen 306 of the conduit 302 and configured to allow airflow through the lumen 306 in a single direction. The valve 314 can comprise a first end portion 314a configured to be secured to the conduit 302, a free, second end portion 302b, and first and second broad surfaces 316a, 316b (collectively “broad surfaces 316”) opposing one another along a thickness t of the valve 314. In some embodiments, the valve 314 comprises a plate, a disc, a flap, or another suitable one-way check valve. The valve 314 can be configured to transition between an open configuration during inspiration (shown in FIG. 3A) and a closed configuration during expiration (shown in FIG. 3B). In some embodiments, the valve 314 is configured to rotate about its first end portion 314a to transition between the open and closed configurations. Additionally or alternatively, the valve 314 can be configured to translate within the lumen 306 and/or deform to transition between the open and closed configurations.

The valve 314 can be configured to transition between the open and closed configurations in response to the patient's respiration. During inspiration, pressurized air travels away from the pressurized air source and through a first region 306a of the lumen 306 in a first direction and contacts the first broad surface 316a of the valve 314 to force the valve 314 into the open configuration. In the open configuration, the valve 314 does not substantially extend across and/or obstruct the lumen 306 so that pressurized air can travel past the valve 314 from the first end portion 302a of the conduit 302 to the second end portion 302b of the conduit 302. The broad surfaces 316 can be substantially parallel to or angled only slightly relative to a longitudinal axis of the conduit 302 in the open configuration. The valve 314 can have a small aspect ratio and/or thickness t such that little energy is required to move the valve 314 into the open configuration and the interaction of the valve 314 with the pressurized air does not significantly change the pressure of the air reaching the patient. The thickness t can be configured to provide pliability to the valve 314 such that the valve 314 behaves like a spring-loaded hinge. The thickness t can also be selected to provide the valve 314 with sufficient structural strength to withstand the exhalation pressure without collapsing. In some embodiments, the valve 314 can be made of a semi-rigid material that is mounted on a hinge that is spring loaded.

During expiration, exhaled air travels away from the patient and through a second region 306b of the lumen 306 in a second direction different than (e.g., opposite to, angled relative to, etc.) the first direction and contacts the second broad surface 316b of the valve 314. The exhaled air imparts a greater force on the second broad surface 316b than the pressurized air imparts on the first broad surface 316a, thus forcing the valve 314 into the closed configuration. In the closed configuration, the valve 314 extends across the lumen 306 to prevent the exhaled air from traveling through the lumen 306 in the second direction beyond the valve 314. In some embodiments, when the valve 314 is in the closed configuration, the broad surfaces 316 of the valve 314 are disposed at a greater angle relative to the longitudinal axis of the conduit 302 than when the valve 314 is in the open configuration. The broad surfaces 316 can be perpendicular or non-perpendicular to the longitudinal axis of the conduit 302 when the valve 314 is in the closed configuration.

The venting device 304 can include a diaphragm 318 positioned within the cavity 310 and configured to control the flow of exhaled air through the outlet 312 in response to changes in the patient's respiration. The diaphragm 318 can have an open end 318a and a closed end 318b and can be retained within the cavity 310 by a retainer 320 maintaining the open end 318a at a desired position within the cavity 310. The cavity 310 comprises a first chamber 322 between the retainer 320 and the first end portion 308a of the wall 308, a second chamber 324 between the closed end 318b of the diaphragm 318 and the second end portion 308b of the wall 308, and an interior region 326 between the closed end 318a of the diaphragm 318 and the retainer 320. The retainer 320 defines one or more apertures 328 fluidically coupling the first chamber 322 to the interior region 326. The first chamber 322 can be configured to be fluidically coupled to the first region 306a of the conduit lumen 306 and the second chamber 324 can be configured to be fluidically coupled to the second region 306b of the conduit lumen 306. Thus, pressurized air can flow from the first region 306a to the first chamber 322 and the interior region 326 and exhaled air can flow from the second region 306b to the second chamber 324.

The diaphragm 318 can be configured to allow airflow out of the outlet 312 during expiration but not inspiration. For example, the diaphragm 318 can be configured to allow the exhaled air to flow out of the outlet during expiration at a pressure substantially equal to that of the pressurized air and prevent the pressurized air from leaking through the outlet during inspiration. During inspiration (see FIG. 3A), pressurized air within the interior region 326 causes the diaphragm 318 to assume a first configuration in which the diaphragm 318 extends along an inner surface 330 of the wall 308 and covers the outlet 312. Thus, during inspiration, pressurized air cannot leak through the outlet 312, as occurs with conventional PAP systems. By preventing pressurized air leakage, the venting device 304 can include more outlets 312 and/or larger outlets 312 to vent larger volumes of exhaled air at a given time while maintaining a desired pressure of the pressurized air.

During expiration (see FIG. 3B), exhaled air travels from the second region 306b of the conduit lumen 306 into the second chamber 324 such that a pressure within the second chamber 324 rises above a pressure within the interior region 326. In response, the diaphragm 318 assumes a second configuration in which the diaphragm 318 does not cover the outlet. The diaphragm 318 can move away from the inner surface 330 and/or away from the outlet 312 when transforming from the first configuration to the second configuration. The diaphragm 318 can be configured to deform, collapse, translate, rotate, or otherwise transform between the first and second configurations. According to various embodiments, the diaphragm 318 can include one or more folds, pleats, wrinkles, creases, tucks, gathers, crimps, or other features that facilitate transformation of the diaphragm 318 between the first and second configurations. The diaphragm 318 can be configured to be positioned within the cavity 310 such that these features are not positioned directly over the outlet 312 and/or are positioned between adjacent outlets 312 to prevent air leakage past the diaphragm 318 at the features. In some embodiments, a volume of the interior region 326 is smaller in the second configuration than in the first configuration. In any case, when the diaphragm 318 is in the second configuration, the exhaled air can flow from through the outlet 312 from the second chamber 324 to the environment, thus venting from the PAP system.

The conduit 302 can define one or more first openings 332 and the wall 308 of the venting device 304 can define one or more second openings 334 each configured to align with a corresponding one of the first openings 332 such that the lumen 306 is fluidically coupled to the cavity 310 via the first and second openings 332, 334. In some embodiments, for example as shown in FIGS. 3A and 3B, the airflow controller 300 includes a channel 336 extending between and fluidically coupling one of the first openings 332 and a corresponding one of the second openings 334. The channel 336 can comprise a tubular structure that is monolithic with the conduit 302 and/or the wall 308 or is formed separately from and later joined to the conduit 302 and/or wall 308. In some embodiments, the airflow controller 300 does not include channels 336 between corresponding first and second openings 332, 334. For example, wall 308 can be configured to be positioned in direct contact with the conduit 302 so that corresponding first and second openings 332, 334 are directly fluidically coupled to one another.

In some embodiments, the conduit 302 comprises at least a portion of a conduit of a PAP system (e.g., conduit 206, etc.). For example, the conduit 302 can comprise the PAP system conduit such that the first end portion 302a of the conduit 302 can be configured to couple to the pressurized air source and the second end portion 302b of the conduit 302 can be configured to couple to the coupler or patient interface. In some embodiments, the conduit 302 is a first conduit and the first end portion 302a is configured to couple to a second conduit extending between the airflow controller 300 and the pressurized air source and/or the second end portion 302b is configured to couple to a third conduit extending between the airflow controller 300 and the patient interface. The conduit 302 can comprise all or a portion of a coupler configured to fluidically couple a PAP system conduit to a PAP system patient interface. In some embodiments, the conduit 302 is integrated into and/or comprises at least a portion of the patient interface. The second end portion 302b of the conduit 302 can be configured to couple to the patient's nostrils and/or mouth, for example. In some embodiments, the conduit 302 can comprise a portion of one or more components of a PAP system defining the primary airflow path from the pressurized air source to the patient.

In some embodiments, the conduit 302 comprises a distinct component formed separately from and configured to couple to one or more components of a PAP system. For example, the first end portion 302a of the conduit 302 can be configured to couple to the second end portion of the conduit of the PAP system and the second end portion 302b of the conduit 302 can be configured to couple to the patient interface such that the conduit 302 is positioned between the PAP system conduit and patient interface. The first and second end portions 302a, 302b of the conduit 302 can each be configured to permanently couple to a corresponding component of a PAP system. In some embodiments, the first and second end portions 302a, 302b of the conduit 302 are configured to releasably couple to a corresponding component of a PAP system, thereby facilitating quick and simple replacement of just the airflow controller 300 instead of the entire PAP system component.

The conduit 302 can comprise a substantially tubular structure. In some embodiments, the conduit 302 comprises multiple tubular structures. As shown in FIGS. 3A and 3B, for example, the conduit 302 can comprise a first shaft 338 extending from a first end portion 338a to a second end portion 338b and defining a first shaft lumen 340 and a second shaft 342 extending from a first end portion 342a to a second end portion 342b and defining a second shaft lumen 344. The first shaft 338 can be positioned at least partially within the second shaft lumen 344 such that the second end portion 338b of the first shaft 338 is positioned between first and second end portions 342a, 342b of the second shaft 342. Accordingly, in some embodiments an outer diameter of the first shaft 338 is no larger than a diameter of the second shaft lumen 344. The first and second shafts 338, 342 can be fixed to one another or can be configured to slide and/or rotate relative to one another.

The airflow controller 300 can include a seat within the conduit lumen 306 for the valve 314 to lie upon while in the closed configuration. For example, as shown in FIG. 3B, the second end portion 338b of the first shaft 338 can form a seat that the valve 314 lies upon in the closed configuration. The end surface at the second end portion 338b of the first shaft 338 can be angled relative to the longitudinal axis of the first shaft 338 so that, when the valve 314 is in the closed configuration, the valve 314 is positioned at a desired angle within the lumen 344 of the second shaft 342. In embodiments in which the conduit 302 comprises a single tubular structure, a seating element configured to provide a seat for the valve 314 can be positioned within the lumen 306 of the conduit 302. In some embodiments, an O-ring, washer, rim, flange, or other annular element can be positioned within the lumen 306 and secured to the conduit 302. Additionally or alternatively, the conduit 302 can include a projection extending inwardly from the conduit 302 into the lumen 306. The projection can be configured to provide a seat for the valve 314 in the closed configuration and can be monolithic with the conduit 302 and/or separately formed from and later secured to the conduit 302. In some embodiments, the conduit 302 does not include a seat for the valve 314. For example, the valve 314 can be maintained at a desired angle within the conduit lumen 306 in both the open and closed configurations by only its attachment to the conduit 302 at the first end portion 314a of the valve 314.

Similar to the conduit 302, the wall 308 of the venting device 304 can comprise a substantially tubular structure. However, as previously noted, the first and second end portions 308a, 308b of the wall 308 can be closed. In some embodiments, for example as shown in FIGS. 3A and 3B, one or more of the second openings 334 can be defined in a side surface of the wall 308. Additionally or alternatively, one or more of the second openings 334 can be defined in the end surface at the first end portion 308a and/or the end surface at the second end portion 308b of the wall 308. Although FIGS. 3A and 3B illustrate the second chamber 524 being configured to be fluidically coupled to the second region 306b of the conduit lumen 306, in some embodiments the second chamber 524 can be configured to be fluidically coupled to an exhalation airflow path that is separate from the primary airflow path defined by the conduit lumen 306. For example, the patient interface can define a portion of the primary airflow path and an exhalation airflow path separate from the primary airflow path. The second region 306b of the conduit lumen 306 can be configured to be fluidically coupled to the portion of the primary airflow path defined by the patient interface and the second chamber 324 can be configured to be fluidically coupled to the exhalation airflow path such that the pressurized air and exhaled air are kept separate from one another throughout the entire PAP system.

Because the venting device 304 does not leak pressurized air during inspiration, the wall 308 of the venting device 304 can define more and/or larger outlets 312 than conventional PAP systems. According to various embodiments, the wall 308 can define one outlet 312, two outlets 312, three outlets 312, four outlets 312, five outlets 312, six outlets 312, seven outlets 312, eight outlets 312, nine outlets 312, ten outlets 312, or more than ten outlets 312. The outlets 312 can be spaced apart about a circumference of the wall 308 and/or spaced apart along a length of the wall 308 between the retainer 320 and the second end portion 308b of the wall 308. One or more of the outlets 312 can have a cross-sectional shape that is substantially circular, rectangular, triangular, polygonal, or irregular. Each of the outlets 312 can have a radial dimension of about 0.1 mm to about 2 mm, or about 0.1 mm to about 1 mm, or greater than 0.1 mm, or less than 1 mm. and x.

The diaphragm 318 can be configured such that a minimal force imparted by the exhaled air and/or a minimal change in pressure within the second chamber 324 is required to transform the diaphragm 318 from the first configuration to the second configuration. The diaphragm 318 can be soft, thin, and/or otherwise easily deformable. In some embodiments, the diaphragm 318 exhibits low friction against the inner surface 330 of the wall 308 so that the diaphragm 318 easily separates from the inner surface 330 when transforming from the first configuration to the second configuration. The diaphragm 318 can comprise, for example, silicone, nitrile, any non-stick film, and other suitable materials. The closed end 318b of the diaphragm 318 can be substantially dome-shaped, substantially conical, or any other suitable shape.

According to various embodiments, a surface area of the diaphragm 318 in the first configuration can be substantially the same as a surface area of the diaphragm 318 in the second configuration. In other words, the diaphragm 318 may not stretch when transforming between the first and second configurations. Stretching of the diaphragm 318 from the second configuration to the first configuration would alter the pressure within the airflow controller 300, thus interfering with the PAP therapy and/or placing additional burden on the pressurized air source to generate pressurized air at a consistent intended pressure. Thus, it can be advantageous to configure the diaphragm 318 to simply collapse or fold upon itself when transforming from the first configuration to the second configuration, rather than stretching to a larger surface area when transforming from the second configuration to the first configuration.

In some embodiments, for example, the retainer 320 is positioned within the open end 318a of the diaphragm 318 and frictionally retains the open end 318a against the wall 308. Additionally or alternatively, the retainer 320 can engage an end surface of the open end 318a of the diaphragm 318. In some embodiments, the retainer 320 is adhered, welded, mechanically coupled to, or otherwise secured to the wall 308 of the venting device 304. The retainer 320 can define one aperture 328 or a plurality of apertures 328 for fluidically coupling the interior region 326 to the first chamber 322. In some embodiments, the retainer 320 defines two apertures 328, three apertures 328, four apertures 328, five apertures 328, six apertures 328, seven apertures 328, eight apertures 328, nine apertures 328, ten apertures 328, or more than ten apertures 328. The apertures 328 can be spaced apart along a radial dimension and/or about a circumferential dimension of the retainer 320. In some embodiments, the retainer 320 comprises a ring with one large central aperture 328. The retainer 320 can comprise any suitable structure that maintains the diaphragm 318 at a desired position within the cavity 310 and allows airflow between the first chamber 322 and the interior region 326.

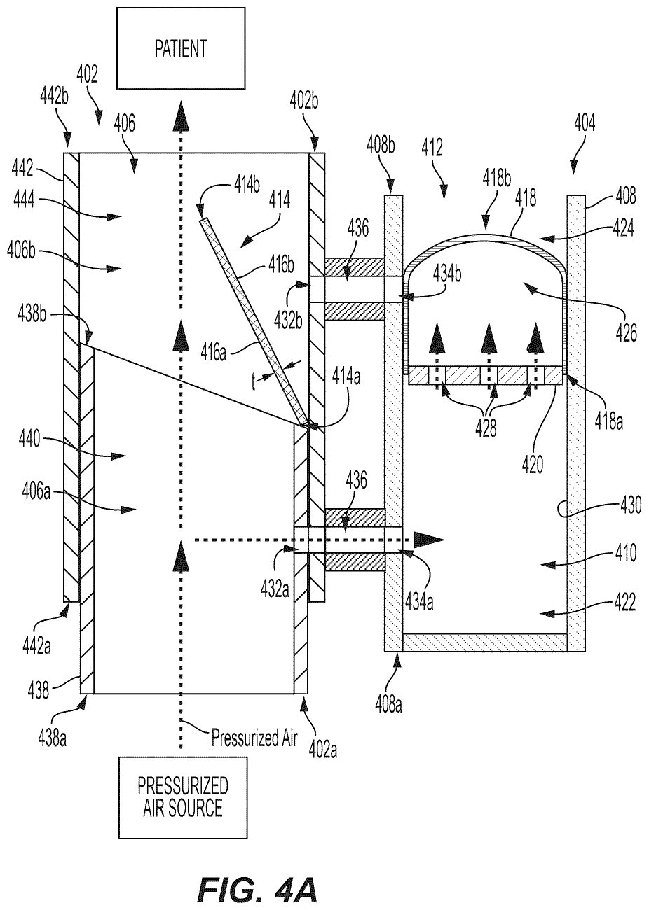

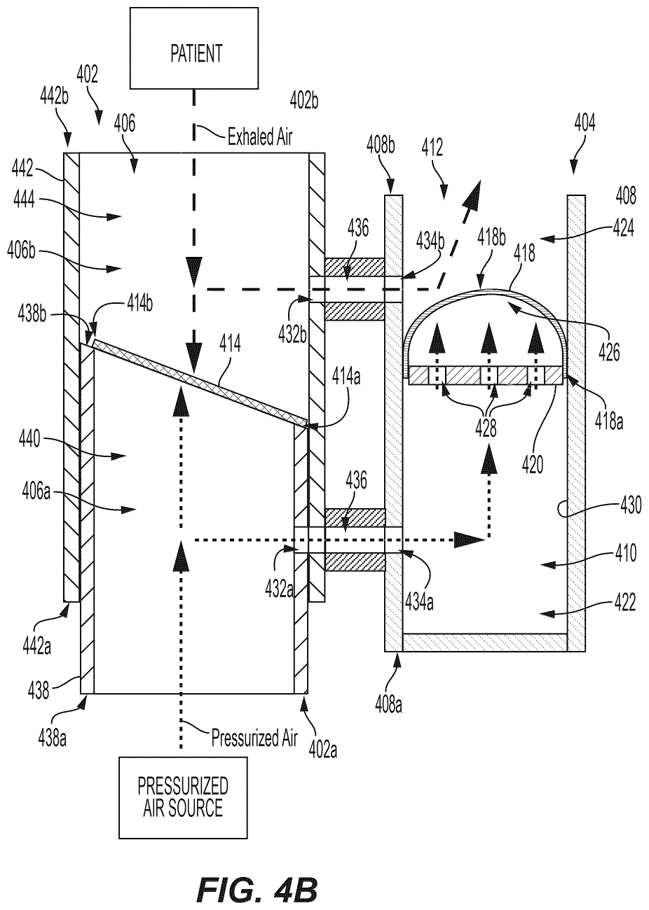

FIGS. 4A and 4B illustrate another airflow controller 400 during inspiration and expiration, respectively, in accordance with various embodiments of the present technology. The features of the airflow controller can be generally similar to the features of the airflow controller 208 of FIGS. 2A and 2B and/or the airflow controller 300 of FIGS. 3A and 3B. With regards to airflow controller 300 and airflow controller 400, like numbers (e.g., conduit 302 versus conduit 402) are used to identify similar or identical components in FIGS. 3A-4B, and the discussion of the airflow controller 400 of FIGS. 4A and 4B will be limited to those features that differ from the airflow controller 300 of FIGS. 3A and 3B. Additionally, any of the features of the airflow controller 400 of FIGS. 4A and 4B can be combined with each other and/or with the features of the airflow controller 300 of FIGS. 3A and 3B.

The airflow controller 400 shown in FIGS. 4A and 4B includes a venting device 404 having a wall 408 with a closed first end portion 408a (similar to closed first end portion 308a of the wall 308) and an open second end portion 408b (in contrast to the closed second end portion 308b of the wall 308). The open second end portion 408b functions as an outlet 412 for exhaled air to vent from the venting device 404 to the environment. During inspiration (see FIG. 4A), the diaphragm 418 is configured to assume a first configuration and prevent airflow into the second chamber 424 of the venting device 404. During expiration (see FIG. 4B), the diaphragm 418 is configured to assume a second configuration and allow exhaled air to flow into the second chamber 424 of the venting device 404. This exhaled air can then pass from the second chamber 424 to the environment via the outlet 412.

As described with reference to FIGS. 3A and 3B, first chamber 422 of the cavity 410 of the venting device 404 is configured to be fluidically coupled to the first region 406a of the conduit lumen 406 and the second chamber 424 of the cavity 410 is configured to be fluidically coupled to the second region 406a of the conduit lumen 406. Specifically, a first one of the first openings 432a is fluidically coupled to the first region 406a and is configured to be fluidically coupled to a first one of the second openings 434a (whether directly or indirectly via a channel 436), which is fluidically coupled to the first chamber 422. Likewise, a second one of the first openings 432b is fluidically coupled to the second region 406b and is configured to be fluidically coupled to a second one of the second openings 434b (whether directly or indirectly via a channel 436), which is fluidically coupled to the second chamber 424.

The interior region 426 of the diaphragm 418 is configured to receive pressurized air from the first chamber 422 via the first region 406a of the conduit lumen 406. As shown in FIG. 4A, during inspiration, the pressurized air within the interior region 426 causes the diaphragm to assume a first configuration in which the diaphragm extends over and prevents airflow through the second one of the second openings 434b. In this manner, the diaphragm 418 prevents the pressurized air from the primary airflow path within the conduit lumen 406 from entering the second chamber 424 and escaping through the outlet 412 of the venting device 404 during inspiration. The airflow controller 400 therefore prevents pressurized airflow leakage and enables the use of a larger outlet 412 for more efficient venting of exhaled air.

During expiration, exhaled air that enters the second region 406b of the conduit lumen 406 closes the valve 414 to prevent the exhaled air from traveling within the conduit lumen 406 past the valve 414. This exhaled air travels through the second one of the first and second openings 432b, 434b. When the pressure of the exhaled air exceeds the pressure of the pressurized air, the exhaled air causes the diaphragm 418 to transform to a second configuration (see FIG. 4B) in which the diaphragm 418 does not extend over or prevent airflow through the second one of the second openings 434b. The exhaled air can therefore flow into the second chamber 424 and out of the venting device 404 via the outlet 412.

Use of the second end portion 408b of the wall 408 as the outlet 412 can enable the outlet 412 to have a large cross-sectional area for efficient venting of exhaled air and is simple to manufacture. Additionally, because the diaphragm 418 need only cover the single second opening 434b fluidically coupled to the second chamber 424, rather than a plurality of outlets distributed along a length of the wall 408 and/or about a circumference of the wall 408, the diaphragm 418 can have fewer pleats, folds, wrinkles, or other features that facilitate transformation of diaphragm 418 proximate to the second opening 434b. Such features, when positioned proximate the second opening 434b and/or outlet(s) can allow pressurized air to inadvertently leak through the second opening 434b and/or outlet(s) and past the diaphragm 418.

Because the open second end portion 408b of the wall 408 functions as the outlet 412, the wall 408 need not define a sidewall outlet along its length between the first and second end portions 408a, 408b (for example as outlet 312 is depicted in FIGS. 3A and 3B). Still, in some embodiments, the wall 408 also defines such a sidewall outlet between the first and second end portions 408a, 408b, in which case the diaphragm 418 can be aligned with the sidewall outlet and configured to cover and expose the sidewall outlet during inspiration and expiration, respectively, as described with reference to FIGS. 3A and 3B.

### Conclusion

Although many of the embodiments are described above with respect to systems, devices, and methods for controlling the flow of a fluid (e.g., air) through PAP systems, the technology is applicable to other applications and/or other approaches, such as controlling fluid flow through mechanical ventilators, mechanical circulatory support systems, and other fluid transport systems, whether medical or non-medical. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-4B.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A device for venting air exhaled by a patient into a lumen of a conduit of a positive airway pressure system, the device comprising:
a wall defining a cavity comprising a first chamber and a second chamber, wherein the wall defines a first inlet fluidically coupled to the first chamber, a second inlet fluidically coupled to the second chamber, and an outlet fluidically coupled to the second chamber, wherein the first and second inlets are configured to be fluidically coupled to the lumen; and
a diaphragm positioned within the cavity and defining an interior region fluidically coupled to the first chamber,
wherein, when a first pressure within the interior region is greater than a second pressure within the second chamber during inspiration, the diaphragm extends across the outlet and prevents airflow through the outlet, and
wherein, when the first pressure is less than the second pressure during expiration, the diaphragm no more than partially extends across the outlet and permits the exhaled air to flow through the outlet from the second chamber.

2. The device of claim 1, wherein the interior region and the first chamber are configured to receive pressurized air from a pressurized air source of the positive airway pressure system.

3. The device of claim 1, wherein the second chamber is configured to receive the exhaled air during expiration.

4. The device of claim 1, wherein the diaphragm is configured to contact an inner surface of the wall proximate the outlet during inspiration.

5. The device of claim 1, wherein the diaphragm is configured to be spaced apart from an inner surface of the wall proximate the outlet during expiration.

6. The device of claim 1, wherein a volume of the interior region is greater during inspiration than expiration.

7. The device of claim 1, wherein the diaphragm is configured to transform between a first configuration during inspiration and a second configuration during expiration.

8. The device of claim 1, wherein the transforming comprises deforming, rotating, and/or translating.

9. The device of claim 1, wherein the wall is substantially tubular.

10. The device of claim 1, wherein the wall has a closed first end and a closed second end opposite the first end along a length of the wall.

11. A device for controlling airflow between a pressurized air source of a positive airway pressure system and a patient, the device comprising:
a conduit defining a lumen extending along a length of the conduit, the conduit having a first end portion configured to be fluidically coupled to the pressurized air source and a second end portion configured to be fluidically coupled to the patient; and
a valve positioned within the lumen and configured to transition between:
an open configuration in which the valve allows pressurized air from the pressurized air source to flow through the lumen from the first end portion of the conduit to the second end portion of the conduit during inhalation; and
a closed configuration in which the valve prevents exhaled air from the patient from flowing through the lumen from the second end portion of the conduit to the first end portion of the conduit during exhalation.

12. The device of claim 11, wherein the valve does not substantially extend across the lumen in the open configuration and does substantially extend across the lumen in the closed configuration.

13. The device of claim 11, wherein the valve is angled relative a longitudinal axis of the conduit by a first angle in the open configuration and a second angle in the closed configuration, and wherein the second angle is greater than the first angle.

14. A device for controlling airflow between a pressurized air source of a positive airway pressure system and a patient, the device comprising:
a conduit having an open first end configured to be fluidically coupled to the pressurized air source and an open second end configured to be fluidically coupled to the patient, the conduit defining a lumen comprising a first region proximate the first end and a second region proximate the second end;
a valve positioned within the lumen between the first and second regions of the lumen;
a wall having a closed first end and a closed second end, the wall defining a cavity comprising a first chamber fluidically coupled to the first region of the lumen via a first inlet and a second chamber fluidically coupled to the second region of the lumen via a second inlet, the second chamber being fluidically coupled to an environment external of the wall via an outlet defined by the wall; and
a diaphragm positioned within the cavity between the first and second chambers,
wherein, during inspiration, a pressure at the first end of the conduit is greater than a pressure at the second end of the conduit, the valve permits pressurized air to flow from the first region of the lumen to the second region of the lumen, and the diaphragm prevents the pressurized air from flowing from the second chamber to the environment via the outlet, and
wherein, during expiration, the pressure at the second end of the conduit is greater than the pressure at the first end of the conduit, the valve prevents exhaled air from flowing from the second region of the lumen to the first region of the lumen, and the diaphragm allows the exhaled air to flow from the second chamber to the environment via the outlet.

15. The device of claim 14, wherein, during inspiration, a pressure within the first region of the lumen and a pressure within the second region of the lumen correspond to the pressure at the first end of the conduit.

16. The device of claim 14, wherein, during expiration, a pressure within the first region of the lumen corresponds to the pressure at the first end of the conduit and a pressure within the second region of the lumen corresponds to the pressure at the second end of the conduit.

17. The device of claim 14, wherein a pressure within the first chamber corresponds to the pressure at the first end of the conduit during inspiration and expiration.

18. The device of claim 14, wherein a pressure within the second chamber corresponds to the pressure at the second end of the conduit.

19. The device of claim 14, wherein during inspiration, the valve extends across the lumen of the conduit, and wherein during expiration, the valve does not extend across the lumen of the conduit.

20. The device of claim 14, wherein during inspiration, the diaphragm extends across the outlet, and wherein during expiration, the diaphragm does not extend across the outlet.

21. A device for venting air exhaled by a patient into a lumen of a conduit of a positive airway pressure system, the device comprising:
a wall defining a cavity comprising a first chamber and a second chamber, wherein the wall defines a first inlet in fluid communication with the first chamber, a second inlet in fluid communication with the second chamber, and an outlet in fluid communication with the second chamber, wherein the first and second inlets are configured to be fluidically coupled to a lumen of a conduit of a positive airway pressure system; and
a diaphragm positioned within the cavity and defining an interior region in fluid communication with the first chamber,
wherein, when a first pressure within the interior region of the diaphragm is greater than a second pressure within the second chamber during inspiration, the diaphragm extends across the outlet and prevents airflow through the outlet, and
wherein, when the first pressure is less than the second pressure during expiration, the diaphragm no more than partially extends across the outlet and permits the exhaled air to flow through the outlet from the second chamber, and allows the first and second pressures to equalize.
